(19) United States
(12) Patent Application Publication
(10) Pub. No.: US 2010/0129297 A1
(43) Pub. Date: May 27, 2010

(54) USE OF AN AQUEOUS GRAPE SEED EXTRACT COMBINED WITH AT LEAST ONE FLUORINE SALT TO COMBAT THE FORMATION OR ACCUMULATION OF DENTAL BIOFILM AND COMPOSITIONS COMPRISING SAID COMBINATION

(76) Inventor: Jean-Claude Vezin, Soual (FR)

Correspondence Address: BIRCH STEWART KOLASCH & BIRCH, PO BOX 747, FALLS CHURCH, VA, 22040-0747, US

(21) Appl. No.: 12/597,114

(22) PCT Filed: Apr. 24, 2008
(86) PCT No.: PCT/EP08/55032

§ 371 Date: Oct. 22, 2009

(30) Foreign Application Priority Data
Apr. 24, 2007 (FR) 07/54670

(51) Int. Cl. A61K 8/69; A61K 36/87; A61Q 11/00; A61P 31/00

(52) U.S. Cl. 424/52; 424/766

(57) ABSTRACT

The invention relates to the use of an aqueous grape seed extract combined with at least one fluorine salt for the preparation of an oral hygiene composition intended to prevent and/or reduce the formation and accumulation of dental biofilm on dental surfaces or on gingival epithelial tissue.

The present invention relates to the use of an aqueous grape seed extract combined with a fluorine salt in order to prevent and/or reduce formation or accumulation of dental biofilm on dental surfaces or the gingival epithelial tissue, as well as associated buccal-dental diseases, while preserving the equilibrium of the buccal ecosystem.

The buccal cavity is a real ecological niche through which transit and dwell saliva, foodstuffs, air and bacterial products.

In the buccal cavity, bacteria may adopt to radically different ways of life: either the one in the planktonic state, in which isolated organisms float in the buccal medium, or the one which in a biofilm, the bacteria attached to a dental surface live as a community.

Dental and periodontal health may be considered as a state of equilibrium in which the bacterial population coexists with the host and where no irreparable damage occurs in the tissues of the latter. However, the disease may occur when the composition and the metabolic activities of the communities of the dental biofilm are perturbed.

Lesions of the periodontium first appear at the gum, causing gingivitises, inflammations of the marginal gum. Next, they may develop into periodontitises affecting the whole of the periodontium and become irreversible in the absence of treatment.

There are different forms of gingivitises:
gingivitis associated with the presence of so-called commonplace negligence plaque,
ulcero-necrotic acute gingivitis,
gingivitis, the origin of which is not related to the plaque:
   associated with skin diseases,
   limited to the gum or affecting the whole of the buccal mucosa;
   allergic;
   infectious.

Dental biofilm (or dental plaque) is a heterogeneous accumulation, adhering to the surface of the teeth and located in the gingivo-dental gap, consisting of a microbial community rich in aerobic or anaerobic bacteria, coated with an intercellular matrix of polymers of microbial and salivary origin. This is a complex bacterial organization, the first formation stages of which correspond to a deposit of glycoproteins on the surfaces of the hard tissues or of the soft tissues lying in the saliva. This first layer bears the name of acquired exogenous film (AEF) or further “salivary biofilm”. It is secondarily colonized by microorganisms which will organize depending on physico-chemical, nutritional or relational criteria, and thereby form a soft, adherent, strong deposit and of a yellowish white color, at the surface of the teeth and of the dental materials currently used.

Two types of plaque are distinguished, defined depending on their anatomic localization relatively to the gum: supra-gingival plaque and sub-gingival plaque. Both of these types of plaques form two different micro-environments. The supra-gingival environment is soaked with saliva, while the sub-gingival environment is soaked with the fluid of the gingival groove. The supra-gingival environment is especially aerobic, while the sub-gingival environment is almost exclusively anaerobic. The sub-gingival space has the shape of a dead end, without any flushing by a liquid, and mechanical forces capable of disaggregating the established bacterial populations are seldom therein. On the contrary, the supra-gingival areas are continually swept by saliva, exposed to all the attrition mechanisms specific to the buccal cavity (mastication, swallowing, phonation) and directly accessible to hygiene measures.

The supra-gingival plaque and the sub-gingival plaque are also distinguished by their pathogenic potential: the supra-gingival plaque is specifically involved in the pathology of caries while the sub-gingival plaque is associated with gingival and periodontal pathologies. The main bacteria found in supra-gingival plaque are Streptococcus mutans, Streptococcus sanguis, Streptococcus mitis, Streptococcus sobrinus, Lactobacillus sp., and Actinomyces sp. (A. viscosus). Depending on its location either on the tooth side or on the epithelium side, the composition of sub-gingival flora varies considerably. The presence of Porphorymonas gingivalis and of Fucobacterium nucleatum is noted, which are thereby strongly involved in periodontal pathologies.

Bactericidal antiseptic agents, are often used in compositions for reducing dental plaque. However, the bactericidal agents are not completely satisfactory since their strong antiseptic action may induce disequilibrium within the natural bacterial population of the buccal cavity. Indeed, antiseptic products act on the microorganisms:
drastically: evaluation of the activity being based on a logarithmic reduction notion, either destruction or deactivation of a high number of microorganisms,
rapidly, by considering a short contact time and a deactivation of most molecules notably in contact with organic materials.

Further, their use over time should be limited because they may promote the selection of resistant strains.

There is therefore a need for developing agents capable of limiting or suppressing the deposit of biofilm by an anti-adhesion action, and thereby allowing preservation of good buccal-dental health without any secondary effects and without perturbing the equilibrium of the buccal ecosystem. This concept is based on an action not at the level of cell growth or viability but at the level of interactions of the bacterial cell with the whole of the buccal surfaces: hard surfaces (for example dental enamel), mucosae, as well as between bacterial cells.

It was surprisingly discovered that such anti-biofilm agents respectful of the equilibrium of the buccal bacterial flora may be extracted from grape seeds.

Grapes contain many active ingredients, including polyphenols. Grape seeds especially contain polyphenols of the oligo-proanthocyanidin (OPC) type which are known for their strong antioxidant power.

Grape seed extracts are indicated in the treatment of venous insufficiency and of varicose veins, for attenuating ocular stress caused by dazzling, or for accelerating the healing of tumefactions consecutive to injury or to surgery.

OPCs of grape seeds are also used in the field of cosmetology as an anti-radical agent for combating skin aging.

It was surprisingly discovered that grape seed extracts may be used in the field of buccal-dental hygiene as anti-biofilm agents.

The inventors moreover discovered surprisingly that the anti-biofilm action of grape seed extracts is potentialized by fluorine salts, such as sodium fluoride or fluorinol (3-pyridyl methanol hydrofluoride).

The object of the present invention is the use of aqueous grape seed extract combined with at least one fluorine salt for preparing a buccal hygiene composition intended to:
prevent the formation or accumulation of dental biofilm on dental surfaces or on the gingival epithelial tissue;
and/or reduce the colonized surfaces.

The fluorine salts according to the present invention are preferably inorganic fluorides, amine fluorides or quaternary ammonium fluorides.

The inorganic fluorides may notably be selected from potassium, sodium and tin fluorides, as well as mixtures thereof.

The amine fluorides are preferably selected from the salts fitting the following formula (I):

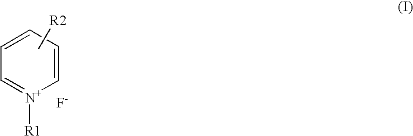

wherein:

R1=H, alkyl;

R2=CH₂—OH, alkyl carboxylate, —CO—NH—CH₂—CH₂—OH,

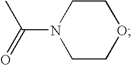

and R2 is found in position 2 or 3.

The aqueous grape seed extract according to the present invention has the characteristic of being rich in polyphenols, notably in proanthocyanidins (OPCs).

The mass fraction of polyphenols in said extract is preferably larger than 50%, preferably larger than 90%.

The mass fraction of proanthocyanidins in said extract is preferably comprised between 20 and 50%.

The mass OPC composition of the extract is preferably the following:
oligomers:
   B1: between 1 and 4%, preferably about 2.5%
   B2: between 5 and 9%, preferably about 7.5%
   B3: up to 3%, preferably about 1.7%
   B4: between 1 and 4%, preferably about 2.6%
monomers:
   catechin: between 3 and 6%, preferably about 4.7%
   epicatechin: between 12 and 15%, preferably about 13.3%
   epicatechin 30 gallate: up to 2%, preferably about 0.7%

The aqueous grape seed extract according to the present invention may be obtained by aqueous extraction with sulfurized water, of white or red grape pomace, preferably white grape pomace. During this extraction, an aqueous solution of sulfur dioxide prepared by dissolving sodium metabisulfite (Na₂S₂O₃) in water and by adjusting the pH to 3.8 with acetic acid may be used as solvent. The following steps may then be carried out:
centrifugation in order to remove the insoluble suspended materials;
filtration in order to notably remove tartaric acid, sugars, polysaccharides and minerals;
evaporation;
subsequently, optionally a new extraction with sulfurized water followed by centrifugation, filtration, evaporation, until a concentrate is recovered as a liquid;
flash pasteurization;
atomization if it is desired that the extract should appear as a powder.

According to the present invention, the aqueous grape seed extract is preferably combined with sodium fluoride or fluorinol (R1=H and R2=CH₂OH in position 3 relatively to the nitrogen). The aqueous grape seed extract is more preferentially combined with fluorinol.

The object of the present invention is also the use of an aqueous grape seed extract in combination with at least one fluorine salt, notably those described earlier for preparing a composition for buccal hygiene intended to:
prevent formation of tartar or occurrence of bucco-dental diseases related to the accumulation of biofilm;
and/or to reduce the colonized surfaces.

Indeed, the composition according to the invention in addition to its preventive effect, may also reduce the plaque index in persons already having a buccal biofilm.

In particular, the combination according to the present invention allows prevention of bucco-dental diseases such as caries, gingivitises or periodontitises, the origin of which is related to accumulation of biofilm.

The anti-adhesion action of the composition is demonstrated by the Applicant:
on an experimental model of buccal biofilm developed from a suspension containing different buccal bacteria on disks of hydroxyl apatite on the one hand;
and by showing the inhibitory activity of said composition on the enzymatic reaction of glucosyl transferases on the other hand.

The object of the present invention is also a composition for buccal hygiene comprising a combination of an aqueous grape seed extract and of at least one fluorine salt, as defined earlier.

Thus, fluorine salts may be selected from inorganic fluorides such as potassium, sodium and tin fluorides, as well as their mixtures, or from amine fluorides, such as those fitting the following formula (I);

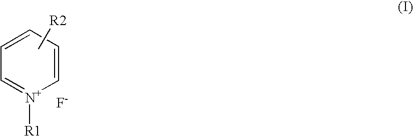

wherein:

R1=H, alkyl;

R2=CH₂—OH, alkyl carboxylate, —CO—NH—CH₂—CH₂—OH,

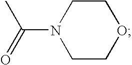

and R2 is found in the position 2 or 3.

The composition according to the present invention preferably contains an aqueous grape seed extract in combination with sodium fluoride or fluorinol (R1=H and R2=—CH₂OH in position 3 relatively to the nitrogen). The aqueous grape seed extract is more preferentially combined with fluorinol.

The composition according to the invention may appear as a mouth wash, a gel or a toothpaste, a gingival gel, a chewing gum, a suckable tablet, dental floss, an extruded capsule, toothbrush bristles, an adhesive bandage, an impregnated towelette, an adhesive paste for dentures and prostheses or a prophylactic polishing paste.

At least one other anti-biofilm such as a cranberry extract (or Vaccinium niacrocarpon) may also be contemplated for combination with the composition according to the present invention.

Said composition may further contain siliglycol and/or vitamin E and/or vitamin C.

Said composition preferably contains between 50 μg/mL and 10,000 μg/mL, advantageously between 200 μg/mL and 5,000 μg/mL, and more advantageously between 1,000 μg/mL and 3,000 μg/mL, and still more advantageously about 2,000 μg/mL of said grape seed extract.

Said composition preferably contains between 50 ppm and 10,000 ppm, more preferentially between 500 ppm and 2,000 ppm; and still more preferentially between 1,000 ppm and 1,500 ppm of fluorine. Advantageously, the composition comprises about 1,500 ppm of fluorine.

Preferably, the composition according to the present invention contains between 1,000 μg/mL and 3,000 μg/mL of the aqueous grape seed extract and between 500 ppm and 2,000 ppm of fluorine, and still more preferably about 2,000 μg/mL of the grape seed extract and between 1,000 and 1,500 ppm of fluorine.

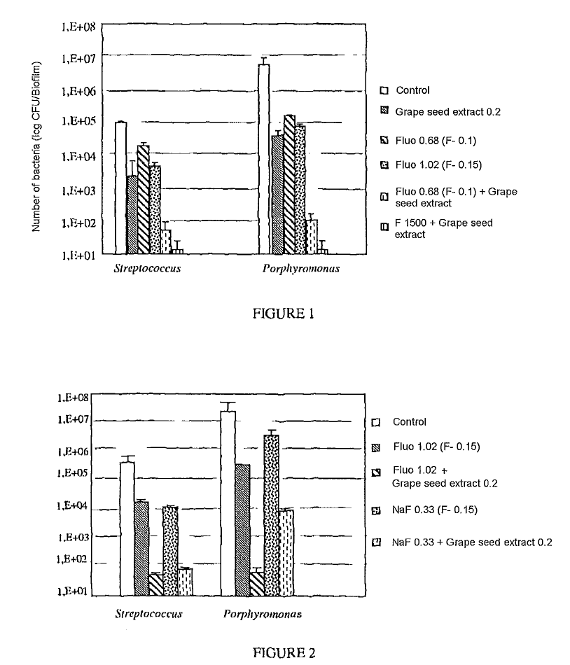

FIG. 1 illustrates the effect of the grape seed extract according to Example 1 in combination with fluorinol on the growth of Streptococci and Porphyromonas gingivalis on an experimental buccal biofilm model in preventive action.

FIG. 2 illustrates the effect of the extract of grape seeds according to Example 1 in combination with NaF on the growth of Streptococci and Porphyromonas gingivalis on an experimental buccal biofilm model in preventive action.

The present invention is illustrated by the following examples.

### EXAMPLES

### 1) Preparation of an Aqueous Grape Seed Extract

The aqueous grape seed extract is provided by the “SociétéFrançaise de Distilleries” (French distillery corporation). It is obtained by extracting with sulfurized water, white grape pomace according to conventional extraction techniques well-known to one skilled in the art.

The characteristics of the extract are the following:
Appearance: fine powder
Color: ochre
Taste: astringent
Density: 0.35-0.55 g/mL
Humidity: <8%

HPLC analysis of the extract gives the following composition:

Oligomers:

| | |
|---|---|
| B1 | 2.56% |
| B2 | 7.51% |
| B3 | 1.72% |
| B4 | 2.61% |

Monomers:

| | |
|---|---|
| Catechin | 4.69% |
| Epicatechin | 13.32% |
| Epicatechin 30 Gallate | 0.66% |
| Total identified OPCs | 33.0% |

### 2) Preventive Action on an Experimental Buccal Biofilm Model

2.1) Procedure

a) Bacterial Strains and Preparation of the Inoculum

The bacterial strains used are:
Streptococcus mutans ATCC 25175
Streptococcus sobrinus ATCC 33478
Porphyromonas gingivalis ATCC 33277

These different bacteria, cultivated in their specific medium, are inoculated into 10 mL of universal culture medium FUM (Fluid Universal Medium, composition of the medium in Annex 1), and then incubated in anaerobiosis at 37° C. for 24 hours.

After checking the purity with a microscope, the series of cultures are independently adjusted to OD₅₅₀ 1.0±0.05 (i.e. about 10⁷ cells per milliliter) by dilutions with FUM. Aliquots (1 mL) of each culture adjusted to the right OD are gathered thereby forming the bacterial suspension used for inoculating the medium from which the biofilm will develop.

b) Preparation of Saliva

Saliva is collected without any stimulation, in healthy volunteers (after obtaining their enlightened consent), at least one hour thirty minutes after having eaten, drunk or having brushed their teeth.

The different collected saliva samples are mixed, centrifuged (30 minutes, 4° C. 15,000 rpm) and the supernatant is pasteurized (30 minutes, 65° C.) and then again centrifuged in sterile tubes. The thereby obtained supernatant is distributed in 50 mL tubes and stored at −20° C. Efficiency of the pasteurization is checked by spreading saliva samples on geloses with blood. After 72 hours of culture at 37° C., no CFU is observed.

c) Preventive Treatment of Biofilms

The biofilm is developed on hydroxyl apatite disks sterilized by heating in the autoclave to 125° C. for 20 minutes.

A. The first step is the formation of the acquired film at the surface of the disks. Each disk is placed in one the wells of a sterile polystyrene 24-well culture dish and incubated with 800 μL of saliva for 4 hours, at room temperature with mild stirring.

B. The saliva is then sucked up from each well and replaced with a mixture containing: 800 μL of saliva+1,000 μL of FUM containing 0.15% of glucose and 0.15% of saccharose+200 μL of the suspension containing the different buccal bacteria (same amount of each).

C. Next, the different amounts of the combinations to be tested are added into the wells in order to obtain the desired concentrations (each concentration is tested in triplicate). See Tables 1 and 2.

TABLE 1
EXPERIMENT No. 1

| | | Final tested |
|---|---|---|
| Products | Added amounts | concentration |
| Control | 153 μL of saline | |
| | (SP) | |
| Extract of grape | 43 μL of grape | 2,000 μg/mL |
| seed according to | seed extract | |
| Example 1 | according to | |
| (solution at 100 mg/mL) | Example 1 + | |
| | 110 μL SP | |
| Fluorinol | 73 μL Fluorinol + | 0.68% Fluorinol |
| (20% solution) | 80 μL SP | (=0.1% fluorine) |
| Fluorinol | 110 μL Fluorinol + | 1.02% Fluorinol |
| (20% solution) | 43 μL SP | (=0.15% fluorine) |
| Grape seed extract | 43 μL of grape | 2,000 μg/mL of |
| according to | seed extract | grape seed extract |
| Example 1 (100 mg/mL) + | according to | according to |
| Fluorinol (20%) | Example 1 + 73 μL | Example 1 + |
| | of Fluorinol + 37 μL | 0.68% Fluorinol |
| | SP | (1,000 ppm |
| | | fluorine) |
| Grape seed extract | 43 μL of grape | 2,000 μg/mL of |
| according to | seed extract | grape seed extract |
| Example 1 (100 mg/mL) + | according to | according to |
| Fluorinol (20%) | Example 1 + | Example 1 + |
| | 110 μL of | 1.02% Fluorinol |
| | Fluorinol | (1,500 ppm of |
| | | fluorine) |

TABLE 2
EXPERIMENT No. 2

| | | Final tested |
|---|---|---|
| Products | Added amounts | concentration |
| Control | 229 μL of saline | |
| | (SP) | |
| Fluorinol | 114 μL Fluorinol + | 1.02% Fluorinol |
| (20% solution) | 115 μL SP | (=0.15% fluorine) |
| Grape seed extract | 45 μL of grape | 2,000 μg/mL of |
| according to | seed extract | grape seed extract |
| Example 1 (100 mg/mL) + | according to | according to |
| Fluorinol (20%) | Example 1 + 114 μL | Example 1 + |
| | of Fluorinol + 45 μL | 1.02% Fluorinol |
| | SP | (1,500 ppm |
| | | fluorine) |
| NaF (4% solution) | 184 μL NaF + 45 μL | 0.33% NaF |
| | SP | (1,500 ppm |
| | | fluorine) |
| Grape seed extract | 45 μL of grape | 2,000 μg/mL of |
| according to | seed extract | grape seed extract |
| Example 1 (100 mg/mL) + | according to | according to |
| NaF | Example 1 + | Example 1 + |
| | 184 μL NaF | 0.33% NaF |
| | | (1,500 ppm of |
| | | fluorine) |

The 24-well dish is then incubated in anaerobiosis at 37° C. for 64 hours.

D. Analysis of the biofilms after 64 hours of incubation. The disks are washed with a solution of saline in order to remove non-adhering bacteria. Each disk is then placed in a sterile Petri dish, and the surface of the disk is scraped with a sterile curette (parodontology instrument). The surface of the scraped disk as well as the Petri dish are rinsed with saline The recovered washing volume is supplemented in order to obtain a final volume of 1 mL and the cell suspension is vortexed.

Serial dilutions to 1:10 of this cell suspension are carried out until concentrations of 10⁻² and 10⁻⁴ are obtained, and then 50 μL of these dilutions are spread over the 4 specific geloses used: mitis salivarius agar, MRS, wilkins chalgren agar (WCA) and blood geloses. The geloses are then placed in anaerobiosis at 37° C. for 48-72 hours and the then the number of formed colonies for each species is counted.

The differentiation of both bacterial species is carried out by observing the morphology of the colonies on gelose, simultaneously with a microscope observation. With the mitis salivarius geloses it is possible to obtain the number of Streptococci and with WCA geloses the number of Porphyromonas.

The number of colonies formed after 48-72 hours of incubation of the molecules with bacterial biofilms is expressed, after counting, in Colony Forming Units per analyzed Biofilm (or CFU/Biofilm) by the formula:

[Number of counted colonies×dilution factor (10² to 10⁴)]/Inoculum volume (0.05 mL)

Next, the average as well as the standard deviation of the three obtained CFU/Biofilm results are evaluated for each concentration of tested molecule and for each group of analyzed bacteria. The graph (logarithmic ordinate axis) is obtained with Excel by taking the average of the three CFU/Biofilm according to the counted bacterium.

2.2) Results/Conclusions

It is observed that the grape seed extract according to Example 1 alone causes a significant reduction larger than 1 log₁₀ of the total number of bacteria in the biofilm developed on the hydroxyl apatite disks. See FIG. 1.

With the grape seed extract according to Example 1, it is therefore possible to limit development of the biofilm.

Moreover, it is observed that the effect of the grape seed extract/fluorinol combination has an activity larger than 3 log₁₀ on Streptococcus and Porphyromonas than that of the extract alone. See FIG. 1.

Fluorinol not only does not interfere with the action of the grape seed extract, but surprisingly and unexpectedly potentializes the effect of the grape seed extract on Streptococcus and Porphyromonas.

It is also observed that the effect of the combination of grape seed extract with NaF reduces the number of bacteria by at least 3 log₁₀ on Streptococcus and Porphyromonas as compared with the extract alone in Experiment No. 1. See FIG. 2.

NaF therefore also potentializes the effect of the grape seed extract on Streptococcus and Porphyromonas.

However, on Porphyromonas, the effect of potentialization of fluorinol is larger than that of NaF.

### 3) Inhibition of Glucosyl Transferases (GTF)

3.1) Procedure

A strain of Streptococcus sobrinus ATCC 33478 is placed in 1 liter of BHI (brain heart infusion) medium enriched in glucose (10 g/L final concentration) in anaerobiosis at 37° C. for 18 hours. This bacterial medium, the pH of which was brought back to 6.5 by means of a NaOH 1N solution, is then centrifuged for 30 minutes at 8,000 rpm at 4° C. The glucosyl transferase, present in the supernatant is concentrated by precipitation with ammonium sulfate. The latter (50% w/saturation v) is gradually added to the supernatant held at 4° C. under stirring for 30 minutes. The mixture is then centrifuged at 8,000 rpm, 4° for 30 minutes. The pellet, rich in glucosyl transferase, is then taken up with phosphate/potassium K₂HPO₄ buffer (10 mM, pH 10.5), dialyzed (MWC 6-8000; width 14.6 mm) for 16 hours and then frozen at −20° C. This is the solution of purified GTF.

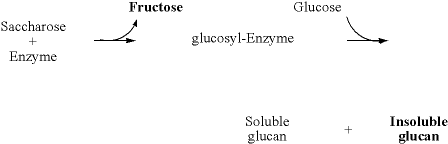

In order to evaluate the action of the different extracts on the enzymatic activity of the glucosyl transferases, two measurements are carried out: the initial reaction rate by assaying the rate of released fructose (assaying of the reducing sugars—DNS test) and the amount of synthesized insoluble glucan (measurements carried out by weighing operations).

The results are expressed as inhibition percentages relatively to control solutions (ultra pure water):
the inhibition percentage of the initial rate of the enzymatic reaction (noted as “fructose” in the following table of results) on the one hand
the inhibition percentage of formation of insoluble glucan produced after 24 hours of incubation at 37° C. (noted as “insoluble glucan” in the following table of results) on the other hand.

The following reaction mixture is placed in an haemolysis tube at 37° C.:

| Glucosyl transferase | Qsp 0.1 to 0.4 | U/mL |
|---|---|---|
| Saccharose | 50 | g/L |
| Phosphate-potassium K₂HPO₄ buffer | 65 mM, pH 6.5 | |
| 100 mM | | |
| Sodium nitride | 0.1 | g/L |
| Dextran T10 | 2 | g/L |
| Polyphenols | 0-2,000 | μg/L |

The reaction may be triggered (t=0) by adding a substrate (saccharose) or an enzymatic solution.

Mother solutions of each extract to be tested are prepared by solubilization of the different powders in mQ water. In each experiment, three control tubes are produced (mQ water) and each concentration extract is tested in triplicate.

Assaying of Reducing Sugars: the DNS Method

Dextran interferes on the whole of the colorimetric methods for assaying proteins, because dextran particles diffract light and generate parasitic absorbance. The amount of glucosyl transferase is therefore measured by its activity under standard conditions. A glucosyl transferase unit represents the amount of enzyme which releases one micromole of fructose per minute.

Activity is determined by measuring the initial production rate of the reducing sugars (fructose) by means of the method with dinitro-3,5-salicylic acid (Sumner and Howell, 1935) with a standard fructose range (0-2.5 g/L).

3,5-dinitrosalicylic acid (DNS) of yellow color is reduced by the reducing carbohydrates in a hot basic medium in order to form a complex of an orange-red color: 3-amino-5-nitrosalicylic acid. With the presented method, the reducing power may be assayed, ranging up to the equivalent of 2.5 g/L of fructose.

The DNS reagent is prepared in the following way; 150 g of sodium potassium tartrate are dissolved in 250 mL of water and then 100 mL of 2N NaOH are added and then while stirring, 1 g of 3,5-dinitrosalicylic acid. mQ water is then added in a sufficient amount for 500 mL.

At times 0 hr and 3 hrs, 200 μL of the reaction medium are sampled and introduced straightaway into haemolysis glass tubes containing 200 μL of DNS (stopping the reaction). The produced reducing sugars are then measured relatively to a standard fructose range (0-2.5 g/L). At the same time, a blank is produced by adding 200 μL of mQ water to 200 μL of DNS.

The tubes are covered with aluminium (DNS is sensitive to light) and brought to 90-100° C. in a water bath for 5 minutes. The whole is then cooled rapidly in ice for 5 minutes. 2 mL of mQ water are then added in all the tubes and the absorbance is read at 540 nm after resting for 20 minutes.

The specific activity is calculated in U/mL of enzyme solution, knowing that 1 U is the amount of enzyme which catalyzes the formation of 1 μmole of fructose per minute under standard conditions.

$$Enzymatic activity ( U \text{/} ml ) = [ ( kinetic assaying curve slope ) / ( Frutose standard curve slope ) ] / ( 180 \times 1 \text{,} 000 \times dilution factor )$$

Assaying of the Amount of Formed Insoluble Glucan

The amount of soluble glucan is measured 24 hours after the beginning of the enzymatic reaction. In order to stop the reaction, the tubes are covered with aluminium and then brought to 90-100° C. in the water bath for 5 minutes. The whole is then cooled rapidly in ice for 5 minutes. The medium (2 mL) is then centrifuged for 30 minutes at 15,000 rpm, at 4° C.

The pellet is then washed twice with ultrapure water, dried for 24 hours at 65° C. and then weighed.

3.2) Results and conclusions

Only fluorinol results in inhibition percentages of the initial enzymatic reaction of 10.3% and 20.6% for concentrations of 0.68% and 1.02% respectively. Further, at both of these concentrations, fluorinol is inefficient on the synthesis of insoluble glucan.

The grape seed extract alone at 0.2% causes 43.9% of inhibition of the initial enzymatic reaction rate and 96.4% of inhibition of insoluble glucan (GI) formation. The addition of 0.68% and 1.02% fluorinol to this 0.2% extract causes better percentages of the inhibition of the initial reaction rate of 57.4% and 65.7%, respectively.

The percentages of inhibition of insoluble glucan synthesis obtained with this combination are 96.1% and 97.1% respectively.

Overall, fluorinol therefore allows potentialization of the grape seed extract activity of inhibiting GTFs.

1. The use of an aqueous grape seed extract in combination with at least one fluorine salt for preparing a buccal hygiene composition intended to prevent and/or reduce the formation and accumulation of dental biofilm on dental surfaces or on gingival epithelial tissue.

2. The use according to claim 1, characterized in that said fluorine salt is selected from salts fitting the following formula (I); 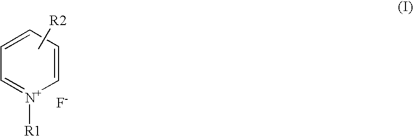
wherein:
R1=H, alkyl;
R2=CH₂—OH, alkyl carboxylate, —CO—NH—CH₂—CH₂—OH, 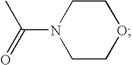
and R2 is found in position 2 or 3.

3. The use according to claim 2, characterized in that said fluorine salt is fluorinol.

4. The use according to claim 1, characterized in that said fluorine salt is sodium fluoride.

5. The use according to any of claims 1 to 4, characterized in that said aqueous grape seed extract contains a mass fraction of polyphenols larger than 50%, preferably larger than 90%.

6. The use according to claim 1, characterized in that said aqueous grape seed extract contains a mass fraction of proanthocyanidins is comprised between 20 and 50%.

7. The use according to claim 1, characterized in that said composition for buccal hygiene is intended for preventing formation of tartar or the occurrence of bucco-dental diseases related to accumulation of biofilm.

8. The use according to claim 7, characterized in that said bucco-dental diseases are selected from caries, gingivitises or parodontitises.

9. A composition for buccal hygiene comprising a combination of an aqueous grape seed extract and of a fluorine salt.

10. The composition according to claim 9, characterized in that the fluorine salt is selected from salts fitting the following formula (I); 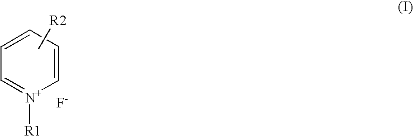
wherein:
R1=H, alkyl;
R2=CH₂—OH, alkyl carboxylate, —CO—NH—CH₂—CH₂—OH, 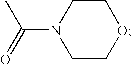
and R2 is found in position 2 or 3.

11. The composition according to claim 10, characterized in that said fluorine salt is fluorinol.

12. The composition according to any of claims 9 to 11, characterized in that it appears as a mouthwash, a toothpaste, a gingival gel, a chewing gum, a suckable tablet, dental floss, an extruded capsule, toothbrush bristles, an adhesive bandage, an impregnated towelette, an adhesive paste for dentures and prostheses or a prophylactic polishing paste.

13. The composition according to claim 9, characterized in that it further contains siliglycol and/or vitamin E.

14. The composition according to claim 9, characterized in that it contains between 50 μg/mL and 10,000 μg/mL, preferably between 200 μg/mL and 5,000 μg/mL, still more advantageously between 1,000 μg/mL and 3,000 μg/mL, and still more advantageously about 2,000 μg/mL of said grape seed extract.

15. The composition according to claim 9, characterized in that it contains between 50 ppm and 10,000 ppm, preferably between 500 ppm and 2,000 ppm of fluorine.
